(19) United States
(12) Patent Application Publication
(10) Pub. No.: US 2009/0147908 A1
(43) Pub. Date: Jun. 11, 2009

(54) Compression of computed tomography data

(76) Inventor: Walter GARMS, Berkeley, CA (US)

Correspondence Address: General Electric Company;GE Global Patent Operation, PO Box 861, 2 Corporate Drive, Suite 648, Shelton, CT, 06484, US

(21) Appl. No.: 11/953,666

(22) Filed: Dec. 10, 2007

(51) Int. Cl. A61B 6/00

(52) U.S. Cl. 378/4

(57) ABSTRACT

Methods, computer-readable mediums, apparatuses, and systems are provided. In one embodiment, the method acquires gain data for a view. The gain data is stored for subsequent use. Thereafter, the method acquires raw data for the view. For detectors in the view, the method compares the raw data to the gain data to determine if the result exceeds a threshold. Generally, information that exceeds the threshold is valid data. The method transmits only the valid data. Embodiments of the invention also include other methods, computer-readable mediums, apparatuses, and systems that contain features similar to the features in the above described method.

### BACKGROUND OF THE INVENTION

1. Field of the Invention

Embodiments of the present invention generally relate to X-ray scanning and more particularly, to methods, computer-readable mediums, apparatuses, and systems for compression of computed tomography data.

2. Description of the Related Art

In some known computed tomography (“CT”) imaging system configurations, an X-ray source projects a fan-shaped or a cone-shaped beam, which is collimated to hit a linear or two-dimensional array of detectors. The X-ray beam passes through an item being imaged. The beam, after being attenuated by the item, impinges upon an array of radiation detectors. Each detector element of the array produces a separate electrical signal that is a measurement of the beam intensity at the detector location. During data acquisition, a gantry rotates around a tunnel with an X-ray emitter. For each gantry position a set of X-ray intensity measurements from all detectors is called a view. The X-ray intensity data in the view is referred to as “raw data.”

Raw data sinograms represent a large amount of data. For a single row of 1000 detectors, and views collected every 0.25 degrees (1440 views), at 16 bits of data per detector, the raw data sinogram would be almost 3 MB. Some scanners may have as many as 32 or 64 rows of detectors, so the total amount of data per rotation could be as great as 180 MB. This large data volume creates problems. For example, the large data volume requires a high transmission bandwidth and incurs additional costs for storage space. Traditional compression algorithms do not work well on raw data for several reasons. Primarily, the reason is that the reconstruction algorithms do not tolerate any loss of information in the part of the scan that contains an item (e.g., a bag). But all of the scan, including parts that do not contain the scanned item, have a large amount of variation. A loss free compression scheme typically does not achieve significant compression, and a lossy compression scheme often loses too much information.

Therefore, there is a need in the art for improved compression of raw data for transfer and/or for storage.

### BRIEF DESCRIPTION

These and other deficiencies of the prior art are addressed by embodiments of the present invention, which generally relates to X-ray scanning systems and more particularly, to methods, computer-readable mediums, and systems for improved compression of raw data for transfer and/or for storage. In one embodiment, the method acquires gain data for a view. “Gain data” as used herein is defined as data that is acquired from an x-ray scan when there is nothing on the conveyor (i.e., nothing on the portion of the conveyor that is surrounded by the gantry). The gain data is stored for subsequent use. Thereafter, the method acquires raw data for the view. For detectors in the view, the method subtracts the raw data from the gain data to determine if the result exceeds a threshold. Generally, information that exceeds the threshold is valid data. The method compresses and transmits the valid data.

In another embodiment, the method acquires gain data from a plurality of detectors for a view. Thereafter, the method also acquires raw data from the plurality of detectors for that view. The method computes a primary ratio, beginning at a first detector in the plurality of detectors, between the gain data and the raw data for each detector until the primary ratio exceeds a threshold. When the threshold is exceeded, a first valid detector position is set. The method then computes a secondary ratio, beginning at a last detector in the plurality of detectors, between the gain data and the raw data until at least one of two events occurs: first, the secondary ratio exceeds the threshold; or second, a detector position associated with the secondary ratio equals the first valid detector position. When either of these two events occurs, the method sets a last valid detector position. Thereafter a view packet is created which includes, in the view packet header, the first and last valid detector position. The valid data, from the first to the last valid detector position, inclusive, is placed in the body of the view packet (or payload of the view packet). The view packet is then transmitted.

In yet another embodiment, a view packet is received. The view packet includes a header. Some of the information in the header defines boundaries for valid data. The header is read to determine a storage location for a normalized view. The view packet body, which contains the valid data, is read. Standard normalization is performed on the valid data. Thereafter the normalized value is set to 0 for the invalid (i.e., non-transmitted) data.

In still another embodiment, a view packet is received. The view packet includes a header. As indicated above, some of the information in the header defines boundaries for valid data. The header is read to determine a storage location for a raw data view. The valid detector data is placed in the raw data view. Thereafter, gain data is placed in the raw data view where there is no valid detector data. Then, the entire raw data view is normalized.

Other embodiments are also provided in which a computer-readable mediums, apparatuses, and a systems perform similar features recited by the above methods.

### BRIEF DESCRIPTION OF THE DRAWINGS

So that the manner in which the above recited features of the present invention can be understood in detail, a more particular description of the invention, briefly summarized above, may be had by reference to exemplary non-limiting embodiments, illustrated in the appended drawings.

FIG. 1 is a perspective view of an exemplary gantry/conveyor combination in accordance with aspects of this disclosure.

FIG. 2 depicts a perspective view of an exemplary emitter and detector array combination in accordance with aspects of this disclosure.

FIG. 3 depicts an embodiment of a block diagram of a system in accordance with aspects of this disclosure.

FIG. 4 depicts an exemplary graph of pixel fluctuation of a scanned item on a conveyor for one view and one detector row.

FIG. 5 depicts an embodiment of a raw data sinogram of an item of a single detector row for about 250 degrees of rotation.

FIG. 6 depicts an embodiment of a cropped raw data sinogram depicted in FIG. 5 in accordance with aspects of this disclosure.

FIG. 7 depicts an embodiment of a high-level block diagram of a method in accordance with aspects of this disclosure.

FIG. 8 depicts an embodiment of a high-level block diagram of another method in accordance with aspects of this disclosure.

FIG. 9 depicts an embodiment of a high-level block diagram of yet another method in accordance with aspects of this disclosure.

FIG. 10 depicts an embodiment of a high-level block diagram of a computer architecture used in accordance with aspects of the invention.

To facilitate understanding, identical reference numerals have been used, wherever possible, to designate identical elements that are common to the figures.

### DETAILED DESCRIPTION

In the following description, numerous specific details are set forth to provide a more thorough understanding of the invention. As will be apparent to those skilled in the art, however, various changes using different configurations may be made without departing from the scope of the invention. In other instances, well-known features have not been described in order to avoid obscuring the invention. Thus, the invention is not considered limited to the particular illustrative embodiments shown in the specification and all such alternate embodiments are intended to be included in the scope of the appended claims.

One of the technical effects disclosed herein is an improved compression of raw data for transfer and/or for storage.

Aspects of the invention are described herein as utilizing an X-ray emitter/detector array combination that rotates. However, it is appreciated that aspects of the invention can be used with scanners that have a stationary X-ray emitter and/or detector array.

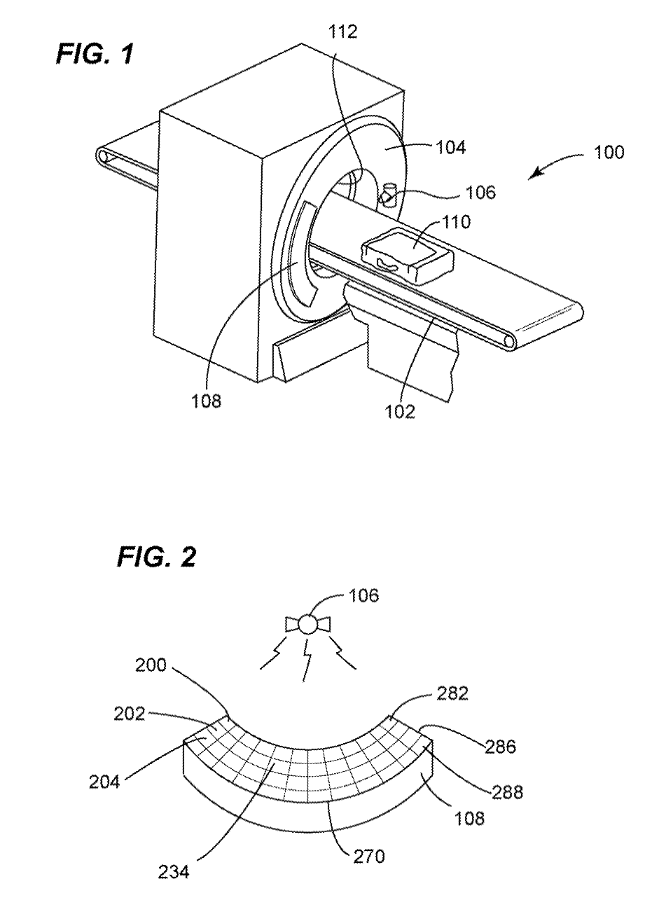

FIG. 1 is a perspective view of a gantry/conveyor combination 100. The gantry/conveyor combination 100 includes a conveyor 102 and gantry 104. The gantry 104 includes an emitter 106 (e.g., an X-ray emitter), a detector array 108, and a gantry tunnel 112. In operation, the conveyor 102 moves such that when an item (e.g., item 110) is placed on conveyor 102, the conveyor 102 moves the item towards; through; and past the gantry 104 and gantry tunnel 112.

The X-ray emitter 106 and the detector array 108 are rotated with the gantry 104 within the imaging plane and around the item(s) to be imaged such that the angle at which an X-ray beam intersects the item 110 constantly changes (hereinafter each change is referred to as “a view”). As the item 110 passes through the gantry 104, the gantry gathers x-ray intensity data (hereafter referred to as “raw data”) acquired from detectors in the detector array 108 for each view.

For exemplary purposes only, each view is about 0.25 degrees from an immediately preceding view and about 0.25 degrees from an immediately subsequent view. Thus (in this example), for a full gantry rotation there can be about 1440 views. The set of raw data views is called a “sinogram.” It is appreciated however, that more views can be acquired (i.e., each view is less that 0.25 degrees apart) and utilized in accordance with this disclosure.

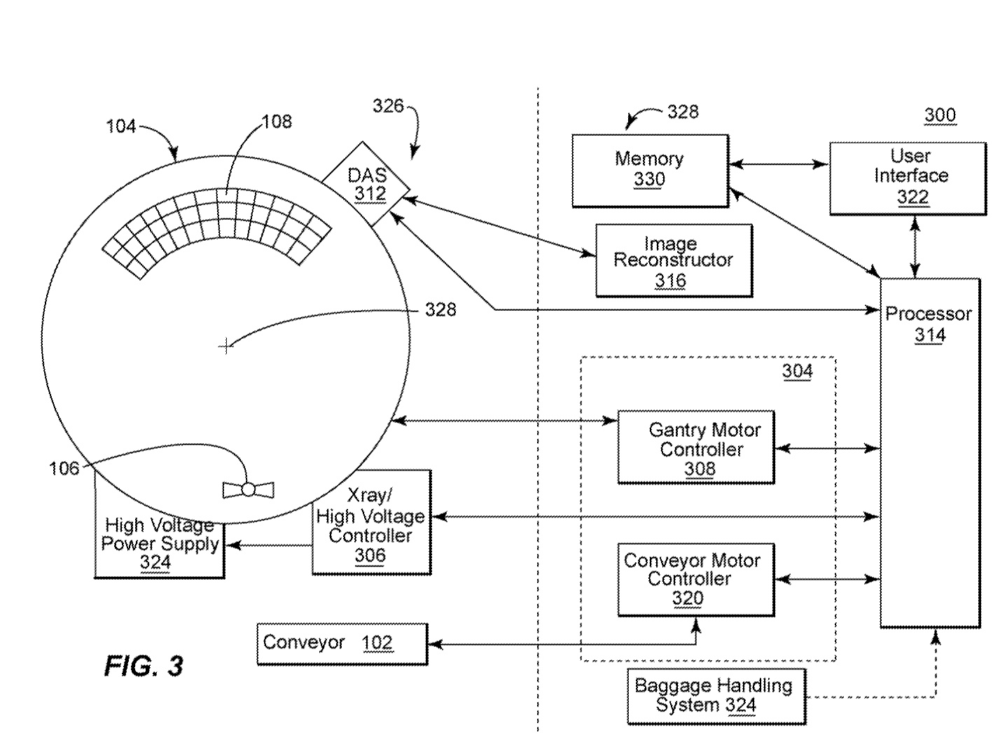

When the gantry 104 gathers raw data for a view, a ratio between gain data and raw data for detectors is acquired for subsequent analysis to determine which detectors contain “valid data.” Thereafter, the valid data is packetized and transmitted towards an image reconstructor 316 (shown in FIG. 3 and explained in greater detail below).

FIG. 2 depicts a perspective view of an embodiment of the emitter 106 and detector array 108. Typically, the emitter 106 emits X-rays that the detector array 108 is designed to detect. The operating principles of the emitter 106 and detector array 108 combination is known and will not be discussed in detail. The detector array 108 has a plurality of detectors (e.g., thousands of detectors). For simplicity, the detector array 108 is described utilizing a few of the detectors (i.e., detectors 200, 202, 204, 234, 270, 282, 286, and 288) in the detector array 108.

FIG. 3 depicts an embodiment of a block diagram of a system 300 used in accordance with aspects of the invention. For clarity, System 300 is separated into moving components 326 and stationary components 328.

The moving components 326 include the gantry 104, conveyor 102, an X-ray/high voltage controller 306, a data acquisition system (“DAS”) 312, and a high voltage power supply. The DAS 312, X-ray/high voltage controller 306, and high voltage power supply 324 are secured to (and rotate in unison with) the gantry 104.

The stationary components 328 include a control mechanism 304, a processor 314, a user interface 322, memory 330, an image reconstruction subsystem 316, and a baggage handling system 324. The control mechanism 304 includes a gantry motor controller 308 and a conveyor motor controller 320.

The gantry 104 includes the emitter 106 and the detector array 108. Each detector (e.g., detectors 200, 202, 204, 234, 270, 282, 286, and 288), shown in FIG. 2, in the detector array 108 produces an electrical signal that represents the intensity of an impinging X-ray beam and hence allows estimation of the attenuation of the beam as it passes through item 110. During a scan to acquire X-ray projection data, gantry 104 and the components mounted thereon rotate about a center of rotation 328.

Rotation of the gantry 104, the operation of X-ray emitter 106, and movement of the conveyor 102 are governed by the control mechanism 304. The control mechanism 304 includes an X-ray controller 306 that provides power to X-ray source 106, a gantry motor controller 308 that controls the rotational speed and position of gantry 104, a conveyor motor controller 320 and a data acquisition system (“DAS”) 312. The rotating gantry includes an X-Ray power supply (not shown), controller 306, and the DAS 312. The remaining components are stationary (e.g., gantry motor controller 308, processor 304, and image reconstructor 316, etc.). Although the image reconstructor 316 and processor 314 are depicted, in FIG. 3, as separate components that depiction is for illustrative purposes only. It is appreciated that in various embodiments, the image reconstructor 316 may be part of the processor 314 (i.e., a single unit).

Prior to scanning an image, the gantry 104 acquires gain data by scanning an empty tunnel 112. The gain data can be stored (for later use) in memory (not shown) located in the DAS 312 and/or in memory 330.

The DAS 312 samples analog data from detector array 108 and converts the data to digital signals for subsequent processing. For example, when the item (e.g., item 110) is placed on the conveyor 102, the digital signals produced when the item is scanned are raw data. However, because of problems associated with transmission of raw data (e.g., high bandwidth, compression of the high bandwidth, storage demands, etc.), ratios between gain data and raw data are computed for a determination of unwanted data (explained in greater detail below). These ratios can be computed at the gantry 104 or at the DAS 312. To prevent unwanted information from being transmitted, the raw data is cropped and sent to an image reconstructor 316.

The image reconstructor 316 receives sampled and digitized X-ray data from DAS 312 and performs high-speed image reconstruction. The reconstructed image is applied as an input to the processor 314, which stores the image in memory 330. Aspects of the invention include a number of ways upon which the image can be reconstructed. For example, the cropped raw data can be sent to image reconstructor 316. In various embodiments, the image reconstructor 316 overlays the cropped raw data onto the gain data. In other embodiments, the gain data is used to fill portions of the image outside of the boundaries of the cropped raw data.

Processor 314 may also receive commands and scanning parameters from an operator (not shown) via the user interface 322 (e.g., a cathode ray tube, a keyboard, a mouse, and/or like device).

The operator can supply commands and parameters via the user interface 322 to instruct the processor 314 to provide control signals and information to the DAS 312, the X-ray controller 306, the gantry motor controller 308, and/or the conveyor motor controller 320.

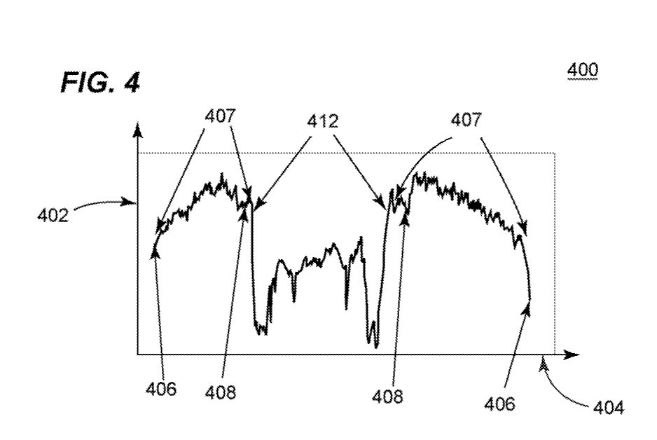

FIG. 4 depicts an exemplary graph 400 of raw data from an item (e.g., a suitcase) scanned on conveyor 102. The graph 400 includes an abscissa “X” axis 404 and an ordinate “Y” axis 402. The “X” axis 404 represents detector numbers for a single row of detectors in array 108 and the “Y” axis 402 represents x-ray intensity.

In the graph 400, lead lines 406 represent the pixel level attributed to the tunnel 112; lead lines 408 represent the pixel level attributed to the conveyor 102; and lead line 412 represents the pixel x-ray intensity attributed to an item of interest (e.g., item 110). Because there is a large amount of fluctuation in x-ray intensity in the unwanted data (e.g. information indicated by lead lines 406 and 408), standard compression techniques will still contain this unwanted information. Aspects of the invention include transmission of the information represented by lead line 412, and compression of all of the remaining data.

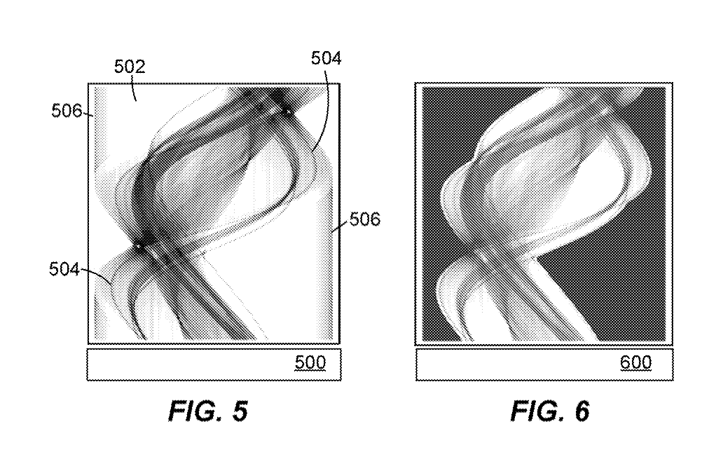

FIG. 5 depicts an embodiment of a raw data sinogram 500 of an item. Lead line 502 represents what is outside of the boundaries of the item (e.g., the air outside of the item, conveyor 102, and tunnel 112). Because detectors collect data at about every 0.25 degrees, raw data sinograms represent a large amount of data. As the number of detectors and rows of detectors increases the amount of data also increases. Note that the amount of data also includes the air outside of the item (depicted by lead line 502); the conveyor 102 (depicted by lead line 504); and tunnel 112 (depicted by lead line 506).

FIG. 6 depicts an embodiment of a cropped raw data sinogram 600. The cropped raw data sinogram 600 represents the information contained in raw data sinogram 500 (depicted in FIG. 5) minus data that is outside the boundaries of the item.

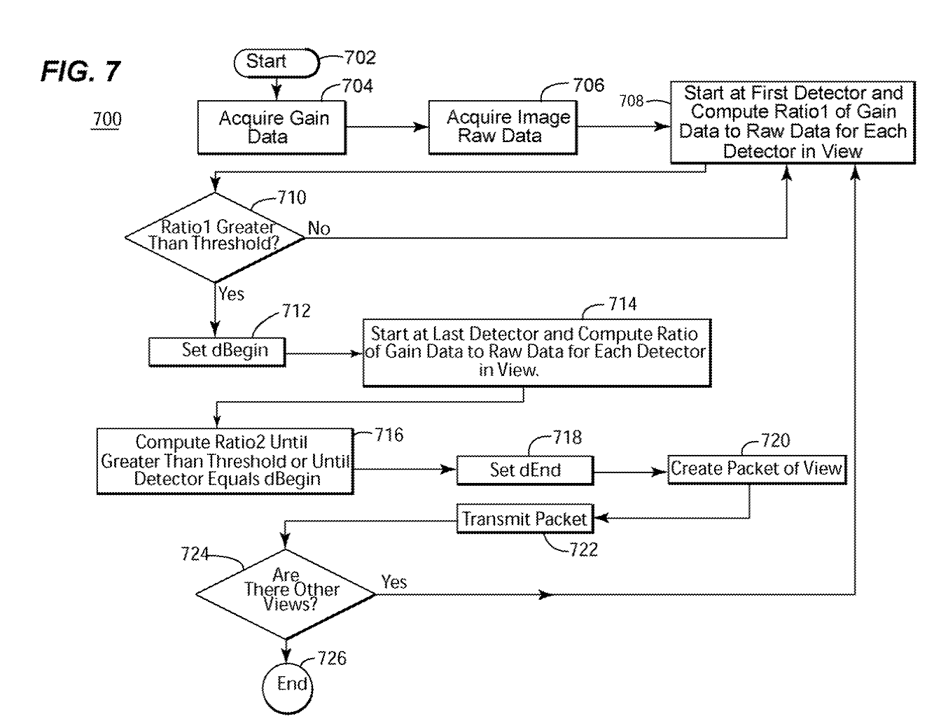

Unless otherwise indicated, the steps described in below (and shown in FIGS. 7, 8, and 9) can be performed in any reasonable order. As such, the description below (and the depiction of FIGS. 7, 8, and 9) is for illustrative purposes only and not intended in any way to limit the scope of the invention.

FIG. 7 depicts an embodiment of a high-level block diagram of a method 700 in accordance with aspects of the disclosure. The method 700 begins at step 702 and proceeds to step 704.

At step 704, the method 700 stores gain data for all views. The gain data is accessed from DAS 312. Each view includes a set of gain data readings from each detector g[0], g[1], g[2], . . . , g[n]. Where g[0] denotes the gain data for the first detector selected in the detector array 108 and g[n] denotes gain data for the maximum number of detectors in detector array 108. For example, referring to FIG. 2, g[0] can be detector 200 and the last possible detector g[n] can be detector 288. The first detector (i.e., g[0] (e.g., detector 200)) selected should be on one end of the detector array 108. Subsequent detectors should be adjacent to the previous detector. For example, g[1] is detector 202, g[2] is detector 204, etc. Thereafter, the method 700 proceeds to step 706.

At step 706, the emitter/detector combination 100 gathers raw data for an item on the conveyor 102 at a view. Illustratively, the raw data is stored in the DAS 312. Raw data is acquired for each detector d[0], d[1], d[2], . . . , d[n]. The annotation of the location of the detectors when collecting raw data corresponds with the annotation of the location of the detectors when the gain data was acquired. For example, gain data from detector 200 is annotated g[0] and raw data acquired from detector 200 is annotated d[0]. Thereafter, the method 700 proceeds to step 708.

At step 708, a ratio between the gain data and raw data for each detector is computed. Specifically, a corresponding view for the gain data and raw data is compared for the detectors. When analyzing this view, the ratio is formed by dividing the gain data of the detector at that view by the raw data of the detector at that same view (i.e., the ratio of g[0] to d[0], g[1] to d[1], etc.). Equation (1) is an exemplary equation which can be used to calculate the ratio and immediately follows:

Ratio1i=gi/di Equation (1)
where Ratio1 is the ratio of gain data to raw data, i represents the position of a detector and begins at i=0, g is the gain data for a detector, and d is the raw data for the same detector.

Although aspects of the disclosure are described herein using Equation (1) it is appreciated that in various embodiments, other equations may be used to calculate the ratio between the gain data and the raw data. For example, the ratio may be calculated using Equation (2) which immediately follows:

Ratio1i=ln((gi−offset)/(di−offset)) Equation (2)
where Ratio1 is the ratio of gain data to raw data, in is the natural logarithm, i represents the position of a detector and begins at i=0, g is the gain data for the detector, and d is the raw data for the same detector. One result of the normalization performed by Equation (2) is that the belt and tunnel are effectively removed from the raw data. Note however, that in embodiments, which utilize Equation (1) above, fewer calculations are performed. For example, there is no need to compute the natural logarithm or consider offset. After calculation of the ratio of gain data to raw data, the method 700 proceeds to step 710.

At step 710, a determination is made whether Ratio1 exceeds a predetermined threshold (e.g., a threshold of about 0.1). The threshold is chosen such that values lower than this threshold represent rays that did not pass through the item of interest. For example, in various embodiments, the threshold is chosen such that data of interest (e.g., all of the data of interest) is included in the un-compressed section. Another factor, which can be used to determine the threshold, is how dense an object needs to be to be considered an item of interest. For example, the threshold can be set so that any part of the item (e.g., a suitcase) generates a value greater than the threshold. If a negative determination is made at step 710, the method 700 returns to step 708 and operates as explained above. If however, an affirmative determination is made at step 710, the method 700 proceeds to step 712.

At step 712, a variable “dBegin” is set to a detector position indicating that the threshold has been exceeded. In various embodiments, dBegin is set to the detector that exceeded the threshold. For example, if Ratio1 for detector 270 has exceeded the threshold then dBegin is set to the location of the detector 270 (i.e., annotated as d[i] where “i” is the sequence number in the iteration of detectors). Although the setting of dBegin to the detector location where Ratio1 has exceeded the threshold is described that description is not intended to limit the scope of the invention.

In other embodiments, when Ratio1 exceeds the threshold dBegin can be set to a prior detector for which a ratio has been calculated. For example, when Ratio1 exceeds the threshold (e.g., at detector 270 where d[i] represents the location of detector 270) dBegin is set to a prior detector (e.g., d[i], d[i−1], d[i−2], or d[i−4]). After dBegin is set, the method 700 proceeds to step 714.

At step 714, a ratio of the gain data and raw data are computed beginning at the last detector (e.g., detector 288) in the detector array 108. An exemplary equation (Equation (3)) for computing the ratio of gain data to raw data follows:

Ratio2i=gi/di Equation (3)

where Ratio2 is the ratio of gain data to raw data, i represents the position of a detector and begins at i=n (where n is the last detector in the detector array 108 (e.g., detector 288)), g is the gain data for a detector, and d is the raw data for the same detector. Although Ratio2 is computed using Equation (3), it is appreciated that other equations (e.g., Equation (2) where i=n and begins at the last detector) may be used in accordance with aspects of this disclosure. After Ratio2 is computed, the method 700 proceeds to step 716.

At step 716, a determination is made whether Ratio2 is greater than a threshold (e.g., a threshold of about 0.1) (“Condition 1”) and/or whether the location of the detector is the location of dBegin (“Condition 2”).

For i=n, if neither Condition 1 nor Condition 2 occurs, Ratio2 is computed for the next detector (where i=n−1). Thereafter, a determination is made whether Ratio2 for the next detector (i.e., at i=n−1) violates either Condition 1 or Condition 2. Step 616 acts as an iterative loop that computes Ratio2 for successive detectors (i.e., i=n−2, n−3, etc.) until either Condition 1 or Condition 2 occurs. When either Condition 1 or Condition 2 occurs, the method 700 proceeds to step 718.

At step 718, a variable “dEnd” is set to a detector position indicating that the threshold has been exceeded. In various embodiments, dEnd is set to the detector that exceeded the threshold. For example, if Ratio2 for a detector d[i=n−20] has exceeded the threshold then dEnd is set to the location of the detector d[i=n−20]. Although the setting of dEnd to the detector location where Ratio2 satisfies Condition 1 and/or Condition 2, that description is not intended to limit the scope of the invention.

In other embodiments, when Ratio2 satisfies Condition 1 and/or Condition 2, dEnd can be set to a prior detector for which a ratio has been calculated. For example, when Ratio2 for d[i=n−20] satisfies Condition 1 and/or Condition 2, dEnd can be set to a prior detector (e.g., d[i=n−19], d[i=n−18], d[i=n−17], or d[i=n−16]). In other words, dEnd=i+1, i+2, i+3, or i+4. After dEnd is set, the method 700 proceeds to step 720.

At step 720, information regarding dBegin and dEnd for the current view is packetized. The view packet has a header, payload, and trailer. The view packet may include information such as, but not limited to, dBegin and dEnd location information in the view packet header. The header may also include the gantry position and conveyor position for that view. Information contained in the range of detectors from dBegin to dEnd is included in the payload; and optionally parity information (e.g., to check data integrity) may be included in the trailer. After step 720, the method 700 proceeds to step 722.

At step 722 the view packet is transmitted to the image reconstructor 316 for analysis, review, and/or analysis. View packet transmission can occur in a variety of ways. For example, transmission can occur via a “slip-ring,” through a wired connection (e.g., a wired connection utilizing stationary detectors); or through a wireless transmission. After transmission at step 722, the method proceeds to step 724.

At step 724, the method queries whether there are other views to be analyzed. There are about 1440 views in a full gantry revolution. If the query is answered affirmatively, the method 700 proceeds to step 708 for analysis of one of the remaining non-analyzed views. If however, the query is answered negatively the method proceeds to and ends at step 726. For spiral scanning systems this process is continuous—at least until the item of interest has been completely scanned. In various embodiments, a determination of the end of the bag is done using optical sensors.

One of the benefits associated herewith is an ability to determine whether an item of interest is in the field of view (i.e., whether valid data found). This condition could be transmitted to the processor to indicate that the item of interest has passed completely through the field of view.

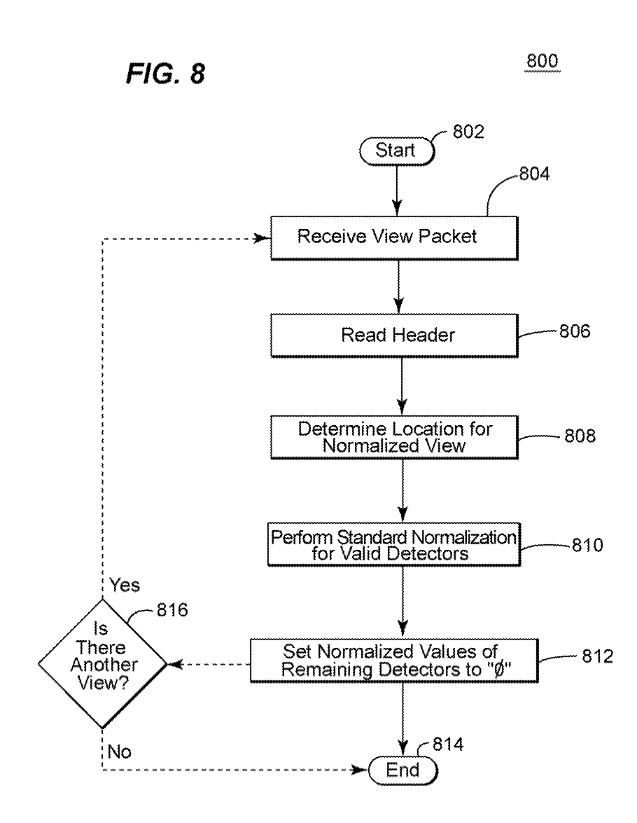

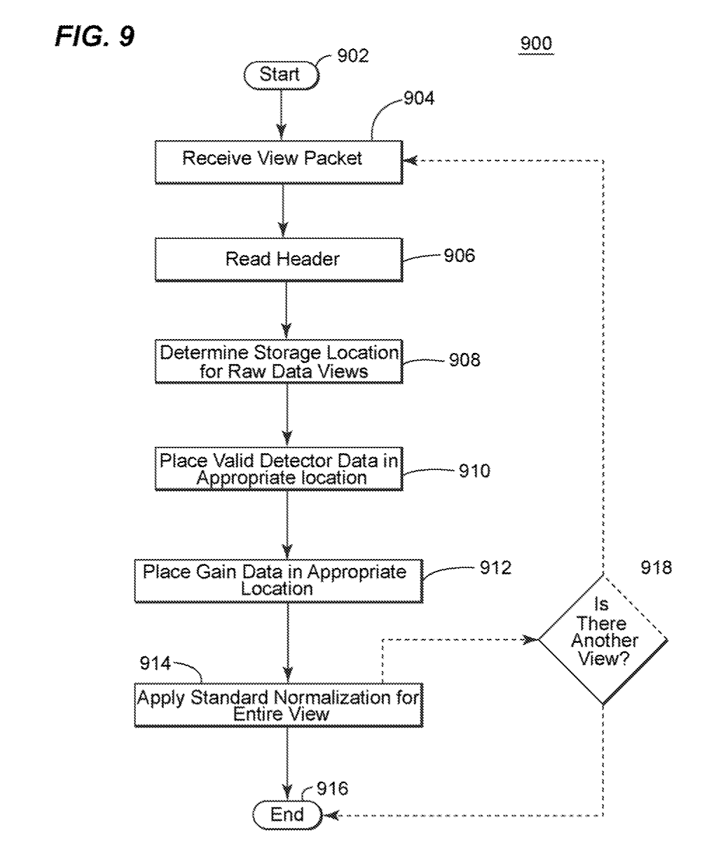

Referring back to FIG. 3, after the image reconstructor 316 receives a data view packet, the image reconstructor 316 analyzes the view packet to aide in reconstruction of an image. FIGS. 8 and 9 depict illustrative embodiments for image reconstruction by the image reconstructor 316.

FIG. 8 depicts an embodiment of a high-level block diagram of a method 800 in accordance with aspects of the disclosure. The method 800 begins at step 802 and proceeds to step 804.

At step 804, the image reconstructor 316 receives a view packet, containing information for a view. The view packet contains a header, a payload, and an optional trailer. Thereafter, the method 800 proceeds to step 806.

At step 806, the image reconstructor 316 reads the header and payload (and optionally, the trailer) of the view packet. After reading the view packet, the method 800 proceeds to step 808.

At step 808, the image reconstructor 316 uses the information contained in the view packet to determine a storage location for a normalized view based on gantry or conveyor position in the header. It is appreciated that in various embodiments, the image reconstructor 316 determines a storage location for the normalized view not based upon the gantry or conveyor position in the view packet header. Thereafter, the method 800 proceeds to step 810.

At step 810, a standard normalization of valid data is performed. In addition to information normally contained in the header (e.g., gantry position, number of detectors, etc.), the header also contains identifiers for the first and last detectors containing valid data (e.g., dBegin and dEnd, respectively). dBegin and dEnd set the boundaries for placement of valid data. With knowledge of the location of dBegin and dEnd it is determined that valid data is also present between dBegin and dEnd. The image reconstructor 316 has the gain data for the view already stored in memory. The view packet payload contains the valid data for the view. The image reconstructor 316 computes standard normalization of the gain data and valid data. Standard normalization can be determined using Equation (2) above. The results are used for image reconstruction. Thereafter, the method 800 proceeds to step 812.

At step 812, the normalized value for all of the remaining detectors that did not have valid data (e.g. for detectors less than dBegin and greater than dEnd) is set to “0.” Thereafter, in various embodiments the method 800 proceeds to and ends at step 814. In other embodiments, after step 812 the method 800 proceeds to optional step 816.

At optional step 816, the method 800 queries whether there are other views. If answered affirmatively, the method 800 proceeds to step 804. If answered negatively, the method proceeds to and ends at step 814.

FIG. 9 depicts an embodiment of a high-level block diagram of a method 900 in accordance with aspects of the disclosure. The method 900 begins at step 902 and proceeds to step 904.

At step 904, the image reconstructor 316 receives a view packet, containing information for a view. The view packet contains a header, a payload, and an optional trailer. Thereafter, the method 900 proceeds to step 906.

At step 906, the image reconstructor 316 reads the header and payload (and optionally, the trailer) of the view packet. After reading the view packet, the method 900 proceeds to step 908.

At step 908, the image reconstructor 316 uses the information contained in the view packet to determine where the raw data should be placed. Specifically, the image reconstructor 316 checks the view packet header. In addition to information normally contained in the header, the header also contains identifiers for the first and last detectors containing valid data (e.g., dBegin and dEnd, respectively). dBegin and dEnd set the boundaries for placement of valid data. With knowledge of the location of dBegin and dEnd it is determined that valid data is also present between dBegin and dEnd. Thereafter, the method 900 proceeds to step 910.

At step 910, the detectors containing valid data (i.e., the first through the last detector containing valid data) are placed in appropriate location. Thereafter, the method 900 proceeds to step 912.

At step 912, gain data is substituted in all detectors positions where no valid raw data was placed in step 910. Thereafter the method 900 proceeds to step 914.

At step 914, a standard normalization is performed on the entire view (for example, using Equation (2)). Thereafter, the method 900, in various embodiments proceeds to and ends and step 916. In other embodiments, the method proceeds to optional step 918.

At step 918, the method 900 queries whether there is another view. If answered in the affirmative, the method proceeds to step 904. If however, a negative determination is made at step 918, the method 900 proceeds to and ends at step 916.

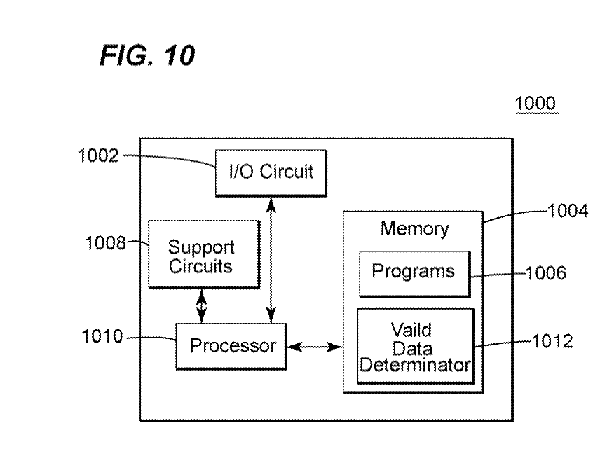

FIG. 10 depicts a high-level block diagram of a general-purpose computer architecture 1000 for performing an embodiment of the invention. For example, the general-purpose computer 1000 is suitable for use in performing the methods of FIGS. 7, 8, and/or 9. The general-purpose computer of FIG. 10 includes a processor 1010 as well as a memory 1004 for storing control programs and the like. In various embodiments, memory 1004 also includes programs (e.g., depicted as a “valid data determinator” 1012) for performing the methods 700, 800, and/or 900. The processor 1010 cooperates with conventional support circuitry 1008 such as power supplies, clock circuits, cache memory and the like as well as circuits that assist in executing the software routines 1006 stored in the memory 1004. As such, it is contemplated that some of the process steps discussed herein as software processes may be loaded from a storage device (e.g., an optical drive, floppy drive, disk drive, etc.) and implemented within the memory 1004 and operated by the processor 1010. Thus, in various embodiments invention, can be stored on a computer readable medium. For example, any/all of the methods 700, 800, and 900 can be stored on computer-readable media as a plurality of instructions which, when executed by a processor, cause the processor the processor to perform any step (or steps) indicated in the methods 700, 800, and/or 900. The general-purpose computer 1000 also contains input-output circuitry 1002 that forms an interface between the various functional elements communicating with the general-purpose computer 1000. For example, in the embodiment of FIG. 7, the general-purpose computer 1000 communicates with the gantry 104 and/or DAS 312 (shown in FIG. 3). The processor 1010 interprets inputs received from the gantry 104 and/or DAS 312 and, in response thereto, the processor 1010 performs method 700, 800, and/or 900. The processor 1010 uses the information acquired from the method 700 to determine valid data the methods 800 and 900 for image reconstruction.

Although FIG. 10 depicts a general-purpose computer 1000 that is programmed to perform various control functions in accordance with the present invention, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein. In addition, although one general-purpose computer 1000 is depicted, that depiction is for brevity only. It is appreciated that the methods 700, 800, and 900 can be in separate computers.

This written description uses examples to disclose the invention, including the best mode, and also to enable any person skilled in the art to make and use the invention. Although specific features of various embodiments of the invention may be shown in some drawings and not in others, this is for convenience only. In accordance with the principles of the invention, each feature may be combined with any or all of the other features in any of the drawings. Further, the patentable scope of the invention is defined by the appended claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method comprising:
acquiring gain data from a plurality of detectors in a view;
acquiring raw data from said plurality of detectors in said view;
comparing said raw data to said gain data to determine valid data;
compressing said valid data; and
transmitting said valid data in a view packet, wherein said view packet comprises at a least a header and a payload.

2. The method of claim 1, wherein said comparison further comprises:
computing a ratio of said gain data to said raw data; and
comparing said ratio to a threshold.

3. The method of claim 1, further comprising:
receiving said view packet;
analyzing said view packet to determine a storage location for a normalized view;
performing standard normalization on valid data; and
setting normalized values of detectors in said plurality not containing valid data to 0.

4. The method of claim 1, further comprising:
receiving said view packet;
analyzing said view packet to determine a storage location for raw data;
placing valid data in said storage location;
placing gain data in remaining locations.

5. The method of claim 1, wherein said comparison further comprises:
computing a primary ratio, beginning at a first detector in said plurality of detectors, of said gain data to said raw data for each detector in said plurality of detectors until said primary ratio exceeds a threshold;
setting a first valid detector position;
computing a secondary ratio, beginning at a last detector in said plurality of detectors, of said gain data to said raw data until at least one of said secondary ratio exceeds said threshold and a detector position associated with said secondary ratio equals said first valid detector position; and
setting a last valid detector position.

6. The method of claim 1, wherein said view packet further comprises a trailer said trailer comprises parity data.

7. A method comprising:
(a). acquiring gain data from a plurality of detectors for a view;
(b). acquiring raw data from said plurality of detectors for said view;
(c). computing a primary ratio, beginning at a first detector in said plurality of detectors, of said gain data to said raw data for each detector in said plurality of detectors until said primary ratio exceeds a threshold;
(d). setting a first valid detector position;
(e). computing a secondary ratio, beginning at a last detector in said plurality of detectors, of said gain data to said raw data until at least one of said secondary ratio exceeds said threshold and a detector position associated with said secondary ratio equals said first valid detector position;
(f). setting a last valid detector position;
(g). creating a view packet; and
(h). transmitting said view packet.

8. The method of claim 7 further comprising repeating (a) through (h) for at least one other view.

9. The method of claim 7, further comprising:
step (i) receiving said view packet;
step (j) analyzing said view packet to determine a storage location for a normalized view;
step (k) performing standard normalization on valid data; and
step (l) setting normalized values of detectors in said plurality not containing valid data to 0.

10. The method of claim 7, further comprising:
(i) receiving said view packet;
(j) analyzing said view packet to determine a storage location for raw data;
(k) placing valid data in said storage location;
(l) placing gain data in remaining locations.

11. A method comprising:
receiving a view packet, said view packet including a header, wherein said header comprises boundaries for valid data;
reading said header;
determining a storage location for a normalized view;
performing standard normalization for said first detector through said last detector; and
setting normalized value of remaining detectors to 0.

12. A method comprising:
receiving a view packet, said view packet including a header, wherein said header comprises boundaries for valid data;
reading said header;
determining a storage location for a raw data view;
placing valid detector data in said raw data view;
placing gain data in said raw data view where there is no valid detector data; and
normalizing all of said raw data view.

13. An X-ray system comprising:
a first subsystem to acquire gain data and raw data from a plurality of detectors;
a second subsystem configured to compare said gain data to said raw data and flag said comparison as valid data when said comparison is greater than a threshold; and
a third subsystem configured to transmit said valid data in a view packet, wherein said view packet comprises at least a header and a payload.

14. The system of claims 13 further comprising:
a fourth subsystem configured to receive and reconstruct said transmitted valid data.

15. The system of claim 14 wherein said reconstruction comprises:
reading said header, wherein said header comprises boundaries for said valid data;
determining a storage location for a normalized view;
performing standard normalization for said first detector through said last detector; and
setting normalized value of remaining detectors to 0.

16. The system of claim 14 wherein said reconstruction comprises:
reading said header, wherein said header comprises boundaries for valid data;
determining a storage location for a raw data view;
placing valid detector data in said raw data view;
placing gain data in said raw data view where there is no valid detector data; and
normalizing all of said raw data view.

17. A computer-readable medium having stored thereon a plurality of instructions, the plurality of instructions including instructions which, when executed by a processor, cause the processor to perform the steps comprising of:
acquiring gain data from a plurality of detectors in a view;
acquiring raw data from said plurality of detectors in said view;
comparing said raw data to said gain data to determine valid data;
compressing said valid data; and
transmitting said valid data in a view packet, wherein said view packet comprises at a least a header and a payload.

18. The computer-readable medium of claim 17, wherein said comparison further comprises:
computing a ratio of said gain data to said raw data; and
comparing said ratio to a threshold.

19. The computer-readable medium of claim 17, further comprising:
receiving said view packet;
analyzing said view packet to determine a storage location for a normalized view;
performing standard normalization on valid data; and
setting normalized values of detectors in said plurality not containing valid data to 0.

20. The computer-readable medium of claim 17, further comprising:
receiving said view packet;
analyzing said view packet to determine a storage location for raw data;
placing valid data in said storage location;
placing gain data in remaining locations.

21. The computer-readable medium of claim 17, wherein said comparison further comprises:
computing a primary ratio, beginning at a first detector in said plurality of detectors, of said gain data to said raw data for each detector in said plurality of detectors until said primary ratio exceeds a threshold;
setting a first valid detector position;
computing a secondary ratio, beginning at a last detector in said plurality of detectors, of said gain data to said raw data until at least one of said secondary ratio exceeds said threshold and a detector position associated with said secondary ratio equals said first valid detector position; and
setting a last valid detector position.

22. The computer-readable medium of claim 17, further comprising:
receiving said view packet;
analyzing said view packet to determine a storage location for a normalized view;
performing standard normalization on valid data; and
setting normalized values of detectors in said plurality not containing valid data to 0.

23. The computer-readable medium of claim 17, further comprising:
receiving said view packet;
analyzing said view packet to determine a storage location for raw data;
placing valid data in said storage location;
placing gain data in remaining locations.

22. A computer-readable medium having stored thereon a plurality of instructions, the plurality of instructions including instructions which, when executed by a processor, cause the processor to perform the steps comprising of:
receiving a view packet, said view packet including a header, wherein said header comprises boundaries for valid data;
reading said header;
determining a storage location for a normalized view;
performing standard normalization for said first detector through said last detector; and
setting normalized value of remaining detectors to 0.
